(19) United States
(12) Patent Application Publication
(10) Pub. No.: US 2017/0231255 A1
(43) Pub. Date: Aug. 17, 2017

(54) Composition Comprising Arthropod Eggs

(71) Applicant: Agrosolutions, Paris (FR)

(72) Inventors: Maxime Ferrero, Bar Sur Loup (FR); Pascal Maignet, Saint Vallier De Thiey (FR); Antoine Bonhomme, Rueil-Malmaison (FR)

(73) Assignee: Agrosolutions, Paris (FR)

(21) Appl. No.: 15/503,160

(22) PCT Filed: Aug. 12, 2015
(86) PCT No.: PCT/EP2015/068588

§ 371 Date: Feb. 10, 2017

(30) Foreign Application Priority Data
Aug. 12, 2014 (FR) 1457771

(51) Int. Cl. A23K 50/90; A23K 20/163; A01G 1/00; A01K 67/033

(52) U.S. Cl. CPC A23K 50/90; A01K 67/033; A23K 20/163; A01G 1/001

(57) ABSTRACT

The present invention concerns a sprayable aqueous composition intended for feeding biological control agents comprising eggs from at least one arthropod species and one or more thickeners and/or gelling agents, and a method for preparing such a composition, the use of same as a nutrient source for biological control agents and for improving the effectiveness of biological control methods, and biological control methods.

The present invention relates to the field of biological control, and especially to biological control products and methods using arthropod biological control agents.

The use of biological control agents constitutes a method for controlling pests, pathogens or weeds by means of their natural antagonistic organisms, such as phytophages (for weeds), parasitoids (for arthropods, etc.), predators (for nematodes, arthropods, vertebrates, molluscs, bats, etc.) or pathogens (for viruses, bacteria, fungi, etc.).

The use of these biological control agents also makes it possible to reduce the use of pesticides.

Typically, the biological control agents are introduced into the crops to be protected in the form of compositions provided in sachets comprising the predators accompanied by food enabling them to feed for a certain period, or else in bulk, in the form of bottles that make it possible to deposit variable quantities of predators on the plants or the soil. The predators then colonize the crops, where they then feed on the targeted prey.

However, in most cases, the biological control agent populations collapse after a certain period of time spent on the plants. It proves necessary, consequently, to reintroduce the predators at regular intervals in order to obtain continuous protection.

Food preparations for feeding biological control agents have been developed in an attempt to circumvent this problem. For example, the application WO2013/48251 discloses gelatin capsules comprising sugar-based compositions, for feeding zoophytophagous insects, for example.

However, these capsules tend to accumulate at the base of the crops, and are consequently difficult to apply near the organic farming agents, which are scattered over the entire plant. Moreover, sugar-based compositions are foods that are not suitable for all the predators commonly used in the field of organic farming.

A need thus still exists for improved means for increasing the lifespan of biological control agent populations in situ, and thus for enabling a sustainable installation of these agents in a greenhouse or a field.

The inventors discovered that compositions based on arthropod eggs, particularly mite, insect or crustacean eggs, and plant gums are particularly effective for improving the lifespan of the predator populations commonly used in the field of biological control.

Thus, the inventors developed compositions that are sufficiently fluid to be sprayed and sufficiently viscous after spraying to coat the leaves and stems of the plants to be treated, while being particularly appealing to predators. Indeed, the fecundity and the longevity of predator populations fed such compositions are much higher than those of untreated populations.

The inventors further showed that similar results can be obtained by preparing the composition of the invention with compounds having properties similar to plant gums, but which are not included in this category. These compounds, selected from thickeners, gelling agents, stabilizers and emulsifiers, make it possible to obtain viscous compositions, which are easily spread over the entire plant.

The present invention has as a first object a sprayable aqueous composition, intended to feed biological control agents, comprising eggs of at least one arthropod species and one or more thickeners and/or gelling agents.

The invention has as a second object a method for preparing a composition according to the invention.

The invention has as a third object the use of a composition according to the invention, as a nutrient source for arthropod biological control agents.

The invention has as another object the use of a composition according to the invention, to improve the effectiveness of biological control methods.

The invention has as another object a biological control method comprising a step of applying on the plants a composition according to the invention.

### FIGURES

FIG. 1. Percentages of viable eggs remaining on pepper seedlings sprayed with Aleuroglyphus ovatus eggs mixed either with 1% by weight acacia gum per volume of water or with water.

FIG. 2. Comparison of mean number of A. swirskii per cucumber seedling with or without spraying of A. ovatus eggs mixed with 1% by weight acacia gum per volume of water as alternative food.

FIG. 3. Comparison of mean number of A. swirskii per pepper seedling with or without spraying of A. ovatus eggs mixed with 1% by weight acacia gum per volume of water as alternative food.

### DEFINITIONS

By “aqueous composition” is meant a composition comprising 10 to 90%, preferably 20 to 80%, particularly preferably 30 to 70%, more preferably 60 to 70% water.

By “sprayable” is meant projectable by crop sprayers generally used in a field, or by manual sprayers used in specialized gardens or crops.

By the expression “egg” is meant, within the meaning of the present invention, collected eggs that have not yet hatched. For example, the eggs can be used fresh or after having been treated by any mode of preservation making it possible to maintain all the nutritive properties of the egg. Preferentially, the eggs are treated by any mode of preservation making it possible to maintain the egg in its developmental stage.

For example, in order to prevent their subsequent development, the eggs can be frozen. Alternatively, in order to prevent them from hatching, the eggs can be specifically treated so as to induce a so-called “sublethal” state. According to the present invention, “sublethal eggs” refer to eggs that cannot hatch but that however retain metabolic activity. A method for obtaining such sublethal eggs is described notably in WO 2013/190142.

By “thickeners and/or gelling agents” is meant any compound, preferably macromolecular, making it possible to reach the viscosity imposed by the expected applications of the composition, i.e., any compound having thickening and/or gelling properties. Apart from these thickening and/or gelling properties, these compounds can play various roles particularly as stabilizers, emulsifiers, water-retaining agents, etc.

According to the invention, the term “dynamic viscosity” refers to viscosity measured by common tools (viscometer), at a temperature of about 20° C. (particularly ±1° C.). Such a viscosity can be measured particularly using a Brookfield-type viscometer at a rotation speed of 5 rpm.

### DETAILED DESCRIPTION

The present invention has as a first object a sprayable aqueous composition, intended to feed biological control agents, comprising eggs of at least one arthropod species and one or more thickeners and/or gelling agents.

According to the invention, the eggs can be whole or fractionated. The eggs can be treated so as to extract particular nutrients.

The eggs of the invention constitute in the true sense an arthropod population; it is thus also possible to define the composition of the invention as comprising:
an arthropod population, said population consisting of eggs of at least one arthropod species,
one or more thickeners and/or gelling agents,
water.

The term “arthropod population” according to the invention means any population comprising at least two individuals belonging to at least one arthropod species, preferably selected from insects, mites or crustaceans.

According to the invention, the arthropod population consists only of individuals still in the egg stage. Thus, within the meaning of the invention, the arthropod population does not comprise arthropods in the larval, nymph or individual adult stage.

According to a preferred embodiment of the invention, the composition comprises, as arthropods, only eggs, and does not comprise arthropods in the larval, nymph or individual adult stage.

Thus, according to the embodiments envisaged by the person skilled in the art, said arthropod eggs are members of one or more mite species, or one or more insect species, or one or more crustacean species. The invention further relates to embodiments wherein said arthropod eggs are members of at least one mite species and at least one insect species, or at least one mite species and at least one crustacean species, or at least one insect species and at least one crustacean species.

The invention also relates to embodiments wherein the arthropod population comprises at least one mite species, at least one insect species, at least one crustacean species.

The invention further relates to embodiments wherein the arthropod population consists of eggs from the same class or the same subphylum. For example, the invention relates to embodiments wherein the arthropod population consists of mite eggs, or insect eggs, or crustacean eggs.

The invention further relates to embodiments wherein the arthropod population consists of a single arthropod species. For example, the invention relates to embodiments wherein the arthropod population consists of eggs of an arthropod species selected from the list consisting of mite species, insect species and crustacean species.

According to an embodiment, the mites are selected from the family Tetranychidae, preferably from the genus Tetranychus.

According to an embodiment, the arthropods are selected from astigmatid mites, for example of the family Pyroglyphidae, the family Acaridae, the family Carpoglyphidae or the family Glyciphagidae.

According to an advantageous embodiment, the astigmatid mites are selected from the family Acaridae, for example the genus Acarus, examples being Acarus siro, Acarus farris, Acarus immobilis or Acarus chaetoxysilos; or according to another example at least the genus Tyrophagus, such as Tyrophagus longior, Tyrophagus similis or Tyrophagus putrescentiae; or the genus Aleuroglyphus, such as Aleuroglyphus ovatus; or the genus Lardoglyphus, such as for example Lardoglyphus konoi; the genus Caloglyphus, such as Caloglyphus mycophagus or Caloglyphus berlesei; the genus Suidasia, such as for example Suidasia nesbitti; the genus Thyreophagus, such as Thyreophagus entomophagus; the family Carpoglyphidae, such as for example the genus Carpoglyphus, for example Carpoglyphus lactis, Carpoglyphus munroi; the family Glycyphagidae, such as for example the genus Glycyphagus, such as Glycyphagus domesticus; the genus Lepidoglyphus, such as Lepidoglyphus destructor; the genus Blomia, such as for example Blomia freemani; the family Chortoglyphydae, the genus Chortoglyphus, for example Chortoglyphus arcuatus.

Advantageously, the astigmatid mite eggs are selected from at least one species of the genus Aleuroglyphus. In a particularly advantageous manner, the astigmatid mite eggs are the species Aleuroglyphus ovatus.

According to an embodiment, the arthropods are selected from insects, in particular Lepidoptera and Diptera, for example the domestic fly (Musca domestica), and Tephritidae (genera Ceratitis, Dacus, Bactrocera).

According to an embodiment of the invention, the Lepidoptera are selected for example from the family Pyralidae, for example Ephestia kuhniella, Ephestia elutella, Ephestia cautella, Corcyra cephalonica or Plodia interpunctella, or for example from the subfamily Gelechiidae, for example Sitotroga cerealella. According to a particularly advantageous embodiment, the Lepidoptera are Ephestia kuhniella eggs.

According to an embodiment, the arthropods are selected from the subphylum Crustacea, for example the class Branchiopoda, the class Cephalocarida, the class Malacostraca, the class Maxillopoda, the class Ostracoda, the class Remipedia.

The composition of the invention comprises one or more thickeners and/or gelling agents. Advantageously, the thickeners and gelling agents used in the composition according to the invention are present in the composition in an amount from 0.01 to 5%, preferably from 0.05 to 4%, preferably from 0.10 to 3%, particularly preferably from 0.2 to 0.5% by weight in relation to the volume of the composition.

For example, the composition according to the invention can comprise 0.01 to 5% w/v gelatin, preferably from 0.02 to 3% w/v, particularly preferably from 0.04 to 2% w/v.

For example, the composition according to the invention can comprise 0.01 to 5% w/v carbomer 940, preferably from 0.2 to 3% w/v, particularly preferably from 0.5 to 2.5% w/v.

For example, the composition according to the invention can comprise 0.01 to 5% w/v maltodextrins, preferably from 0.02 to 4% w/v, particularly preferably from 0.03 to 3.5% w/v.

For example, the composition according to the invention can comprise 0.01 to 5% w/v glucomannans, preferably from 0.05 to 4% w/v, particularly preferably from 0.1 to 3% w/v. For example, the composition according to the invention can comprise 0.01 to 5% w/v exogenous production of Sclerotium rolfsii, preferably from 0.1 to 3% w/v, particularly preferably from 0.25 to 2.50% w/v.

For example, the composition according to the invention can comprise 0.01 to 5% w/v acacia gum, preferably from 0.02 to 4% w/v, particularly preferably from 0.05 to 3% w/v.

Advantageously, the thickeners and/or gelling agents are present in the composition according to the invention in a quantity that provides the composition with a dynamic viscosity at zero velocity gradient between 0.005 and 250 Pa·s, in particular between 0.01 and 100 Pa·s, in particular between 0.02 and 50 Pa·s, preferably between 0.03 and 20 Pa·s, more preferably between 0.04 and 10 Pa·s, particularly preferably between 0.05 and 5 Pa·s.

In particular, the thickeners and/or gelling agents are present in the composition according to the invention in a quantity that provides the composition with a dynamic viscosity at zero velocity gradient between 0.05 and 100 Pa·s, preferably between 0.1 and 50 Pa·s, more preferably between 0.2 and 10 Pa·s, particularly preferably between 0.5 and 1 Pa·s.

The composition according to the invention, once it is prepared, has a viscosity suitable for spraying and then, once it is sprayed, its viscosity increases to adhere to that onto which it has been sprayed, for example the leaves and stems of plants.

The thickeners and gelling agents used in the composition according to the invention preferentially are not thickeners and gelling agents that must be used hot, in order to avoid any risk of damaging the eggs contained in the composition.

By “thickeners and gelling agents that must be used hot” is meant, within the meaning of the present invention, thickeners and gelling agents the use of which involves a heating step, i.e., above 40° C., particularly above 35° C., during the preparation of the compositions according to the invention, in the presence of eggs of at least one arthropod species. Such a thickener or gelling agent is notably agar-agar.

The thickeners and gelling agents used in the composition according to the invention preferentially are not mineral-type thickeners and gelling agents, such as silica gel. Such thickeners and gelling agents are less advantageous since that they solidify over time.

The thickeners and gelling agents used in the composition according to the invention are preferentially glucidic in nature.

The thickeners and gelling agents used in the composition according to the invention are preferentially of plant origin; particularly preferably they are plant gums.

By “thickeners and gelling agents of plant origin” is meant:
plant exudates, such as for example gum arabic, gum tragacanth, karaya gum,
galactomannans derived from certain seeds such as for example guar gum, carob gum, tara gum, oat gum,
glucomannans such as konjac glucomannan,
pectins located within cell walls,
algae extracts, such as for example carrageenans from red algae or alginates from brown algae,
natural or modified starches,
cellulose derivatives, such as for example hydroxypropylcellulose, hydroxypropylmethylcellulose, carboxymethyl cellulose sodium salt,
gels derived from metabolic production of plants such as for example products of exogenous production of Sclerotium rolfsii,
maltodextrins such as for example corn maltodextrins, tapioca maltodextrins, potato maltodextrins,
acacia gum, xanthan gum, gellan gum, gum ghatti, konjac gum, cassia gum.

By “plant gum” is meant, within the meaning of the invention, an adhesive glucidic compound produced inside plant tissues by specialized cells. Plant gum is a viscous substance exuded by certain plants, which can consequently be easily collected. Plant gums are well-known for their thickening, stabilizing, gelling and/or emulsifying properties.

The plant gums within the meaning of the invention are preferentially selected from the list consisting of acacia gum, carob gum, oat gum, guar gum, gum tragacanth, gum arabic, xanthan gum, karaya gum, tara gum, gellan gum, gum ghatti, konjac gum, cassia gum.

The inventors further discovered that the compound of the invention can also be selected from compounds not included in the category of plant gums, but nevertheless having satisfactory thickening or gelling properties.

The thickeners and gelling agents used in the composition according to invention can also be selected from gelatin, chitosan, carbomer 940, styrenes, polyvinylpyrrolidone, isododecane, bis-vinyl dimethicone/dimethicone copolymer, sodium acrylate, acryloyldimethyl/taurate copolymer, isohexadecane, polysorbate, maltodextrins, in particular corn maltodextrins, tapioca maltodextrins, potato maltodextrins, carboxymethyl cellulose sodium salt, glucomannan, psyllium seed or integument powder, products of exogenous production of Sclerotium rolfsii, xanthan, hydroxypropylcellulose.

The thickeners and gelling agents used in the composition according to the invention can also be selected from the list consisting of gelatin, chitosan, carbomer 940, styrenes, polyvinylpyrrolidone, isododecane, bis-vinyl dimethicone/dimethicone copolymer, sodium acrylate, acryloyldimethyl/taurate copolymer, isohexadecane, polysorbate, psyllium seed or integument powder, xanthan gum.

Advantageously, the thickeners and/or gelling agents of the composition of the invention are selected from the list consisting of gelatin, carbomer 940, maltodextrins, for example tapioca maltodextrins, glucomannans, for example konjac glucomannans, gels derived from metabolic production of plants such as exogenous production of Sclerotium rolfsii, plant gums such as acacia gum.

The composition of the invention can further comprise other compounds of interest.

For example, the composition of the invention can comprise additional nutrient sources, in addition to the arthropod eggs, such as for example pollen or vitamins.

The composition of the invention can also comprise antioxidants or preservatives.

For example, but in no way limiting the object of the invention, the antioxidants can be selected from the antioxidants used in the food processing industry, such as for example from the list consisting of ascorbic acid or vitamin C (E300), sodium ascorbates (E301), calcium ascorbates (E302), 5-6-1-diacetyl ascorbic acid (E303), 6-1-palmityl ascorbic acid (E304), citric acid (E330), sodium citrates (E331), potassium citrates (E332), calcium citrates (E333), tartaric acid (E334), sodium tartrates (E335), potassium tartrates (E336) and sodium potassium tartrates (E337), butylated hydroxyanisole (E320), butylated hydroxytoluene (E321), octyl gallates (E311), dodecyl gallates (E312), sodium lactates (E325), potassium lactates (E326), calcium lactates (E327), lecithins (E322), natural tocopherols (E306), synthetic alpha-tocopherol (E307), synthetic gamma-tocopherol (E308), synthetic delta-tocopherol (E309), and all the tocopherols constituting vitamin E.

According to the invention, the preservatives can be selected from food preservatives, in particular selected from the list consisting of nitrates and nitrites (E249-E252), sulfites (E221-E228), sulfur dioxide (E220), hydrogen peroxide, sorbic acid (E200), benzoic acid (E210), fumaric acid (E297), sodium erythorbate (E316), parabens (E214-E219), dimethyl dicarbonate (E242).

Another object of the invention relates to a method for preparing a composition according to the invention.

This method comprises the successive steps of:
dissolving the thickener(s) and/or gelling agent(s) in an aqueous base, preferably in water,
adding the arthropod eggs to this mixture.

The composition of the invention is particularly advantageous for feeding the predators commonly used in the field of biological control, whether in the context of raising these predators, or subsequently, in a greenhouse or a field, after they have been spread over the crops to be treated.

Thus, another object of the invention is the use of the composition of the invention as a nutrient source for biological control agents.

The inventors established that the composition of the invention makes it possible to increase the lifespan and the reproduction of biological control agents, in the context of raising said agents, but also subsequently, in a greenhouse or field, after said agents have been spread over the crops to be treated. The use of the composition of the invention consequently makes it possible to decrease the spreading frequency of biological control agents, indeed to limit it to only one release at the beginning of cultivation, and generally, to improve the effectiveness of biological control methods.

Thus, another object of the invention is the use of the composition of the invention to improve the effectiveness of biological control methods.

The inventors determined in particular that the composition of the invention is particularly attractive for feeding particular arthropod biological control agents. Indeed, these types of predators were observed to grow better when they are fed the composition of the invention.

According to an embodiment, the biological control agents are selected from the class Arachnida, preferentially from mites (order Acari), or from the class Insecta.

According to an embodiment, the mites can be selected for example from the families Phytoseiidae, Macrochelidae, Laelapidae or Cheyletidae.

Advantageously, the Phytoseiidae can be selected from the subfamily Amblyseiinae, for example Amblyseius swirskii, Amblyseius largoensis, Amblyseius andersoni; or from the genus Neoseiulus, e.g., Neoseiulus womersleyi, Neoseiulus californicus, Neoseiulus cucumeris, Neoseiulus fallacis, Neoseiulus longispinosus; or from the genus Iphiseius, e.g., Iphiseius degenerans; or from the genus Amblydromalus, for example Amblydromalus lailae, Amblydromalus limonicus or Amblydromalus manihoti, or from the genus Phytoseiulus, for example Phytoseiulus persimilis, Phytoseiulus macropilis or Phytoseiulus longipes; or from the subfamily Typhlodrominae, in particular the genus Typhlodromips, for example Typhlodromips montdorensis; or from the genus Euseius, for example Euseius ovalis, Euseius scutalis, Euseius finlandicus, Euseius gallicae, Euseius stipulatus, Euseius tularensis, Euseius hibisci.

Advantageously, the mites of the family Macrochelidae can be selected from Macrocheles, for example Macrocheles robustulus.

Advantageously, the mites of the family Laelapidae can be selected from Hypoaspis, for example Gaeolaelaps aculeifer or Stratiolaelaps scimitus.

Advantageously, the mites of the family Laelapidae can be selected from Cheyletus, for example Cheyletus eruditus.

Preferentially, the mites are selected from Amblyseius swirskii, Neoseiulus cucumeris, Amblyseius largoensis, Neoseiulus californicus, Neoseiulus fallacis, Neoseiulus longispinosus, Amblydromalus lailae, Typhlodromips montdorensis, Euseius ovalis, Euseius scutalis, Euseius hibisci. According to a particularly advantageous embodiment of the invention, the mites are the species Amblyseius swirskii.

The invention also relates to a biological control method comprising a step of applying on the plants a composition according to the invention.

The composition provides a nutrient source to the biological control agents already present in the crops.

Thus, the composition of the invention is applied to the plants for the purpose of feeding a biological control agent that was introduced beforehand or that is introduced concomitantly.

Thus, according to this embodiment, the biological control method of the invention comprises the successive or concomitant steps of:
a) introducing biological control agents into the crop;
b) applying a composition according to the invention to the plants of said crop.

Advantageously, the composition according to the invention is applied again, at a maximum of twice per month, preferably once per month, after its first application. Thus, the biological control agents introduced once at the beginning of the method according to the invention are maintained by a constant nutrient source.

The application of the composition according to the invention can be carried out on any type of crop, in particular on greenhouse crops and on field crops. Indeed, the composition of the invention makes it possible to treat plants irrespective of the cultivation conditions.

The application can advantageously be carried out using a sprayer, in particular using a crop sprayer, or by manual sprayers used in specialized gardens or crops.

Once applied, the sprayable composition according to the invention becomes viscous.

The following examples illustrate the invention without limiting its scope.

### EXAMPLES

### Example 1

Astigmatid Mite Eggs Mixed with a Sprayable Preparation Resist Desiccation Better that Astigmatid Mite Eggs Mixed with Water

### Protocol

A solution of acacia gum in water, in an amount of 1% by weight acacia gum/volume of water, is prepared.

Mite eggs of the species Aleuroglyphus ovatus, previously preserved at +3° C. for 6 weeks, were mixed with a preparation of 1% by weight acacia gum per volume of water.

The solution obtained was sprayed using a small sprayer (200 ml) on the underside of leaves of four 25 cm pepper seedlings disposed in a greenhouse. At 7 and 12 days after spraying, a leaf was taken from each plant and the number of collapsed and turgescent eggs were counted.

A thermohygrometer was also disposed within the device to evaluate the extreme temperatures and hygrometries to which the eggs were subjected.

### Results

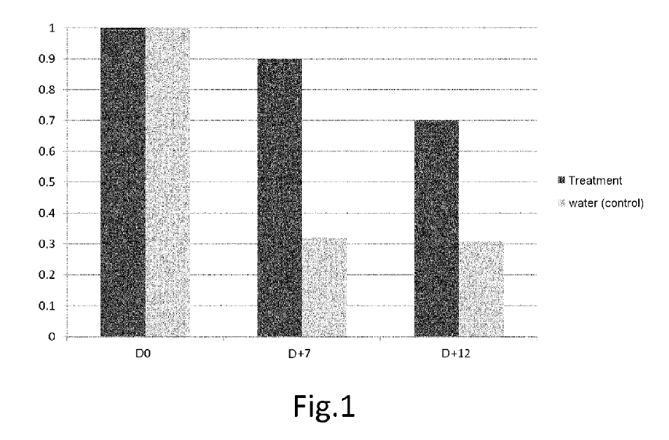

The percentages of viable eggs remaining on the leaves, derived from the means of the egg counts carried out, are presented in FIG. 1 (control=water).

The minimum and maximum temperatures and relative humidities observed in the device over the 12 days of the experiment varied from 9 to 36° C. and from 13 to 93% relative humidity (RH), respectively.

### CONCLUSION

FIG. 1 shows that eggs mixed with a sprayable preparation that becomes viscous after application collapse less quickly than eggs mixed with water, and that they thus resist desiccation better, under highly variable temperature and humidity conditions.

After 12 days of exposure, 70% of the eggs indeed remain viable, whereas only 30% of the eggs mixed with water are still turgescent.

### Example 2

Appeal to Predatory Mites of Arthropod Eggs Mixed with a Sprayable Fluid Preparation

### Protocol

Thirty-six cucumber seedlings and 36 pepper seedlings were planted in a greenhouse, in soilless substrate blocks, with controlled irrigation. Three seedlings were planted per block.

A solution of acacia gum in water, in an amount of 1% by weight acacia gum/volume of water, is prepared.

At DO, every other block (3 plants sprayed, 3 plants not sprayed) was sprayed with a solution according to Example 1 containing A. ovatus eggs and 1% by weight acacia gum/volume of water. Five A. swirskii females were then deposited on each plant (sprayed or not). Next, the plants having been previously sprayed were sprayed again at D+15 and D+30.

A count of the number of A. swirskii per leaf was carried out twice per week, on 5 leaves (pepper) or 3 leaves (cucumber) per plant, for 35 days.

### Results

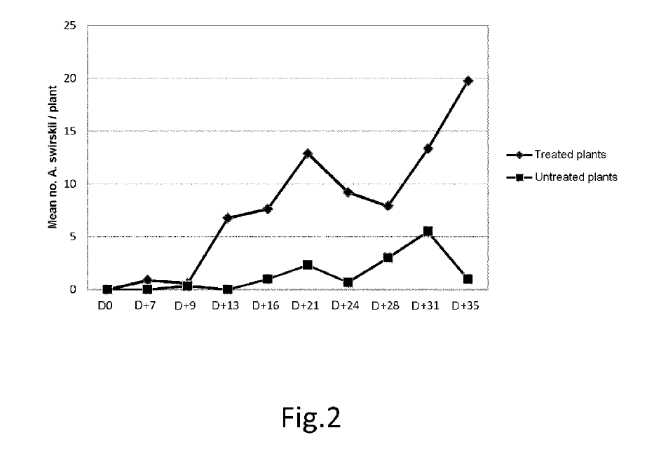

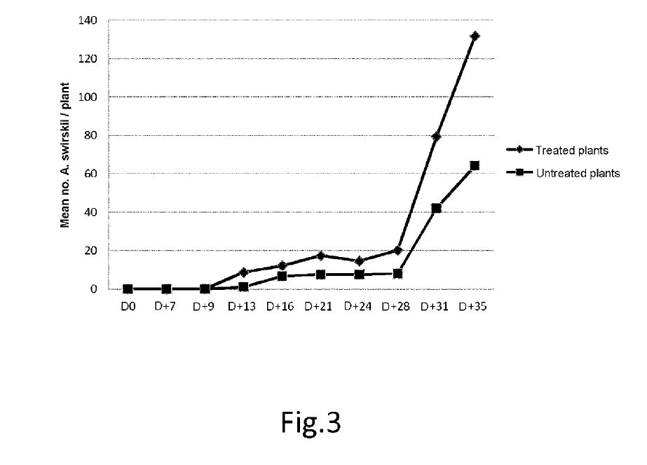

The number of A. swirskii per pepper or cucumber seedling derived from the counts carried out are presented in FIGS. 2 and 3, respectively.

### CONCLUSIONS

Results show that A. swirskii is able to feed and to reproduce in greenhouse cultivations with a fluid but viscous composition containing A. ovatus eggs, as FIGS. 2 and 3 attest, which show an increase in the A. swirskii populations on the treated plants, irrespective of the crop.

On a pepper crop (FIG. 3), in the absence of alternative food, A. swirskii is able to be maintained by feeding on the pollen of the plant, up to levels reaching more than 60 individuals/plant. In the presence of A. ovatus eggs mixed with the acacia gum solution, this installation is not only faster (better preventive effect), but the number of individuals per plant can go up to more than 130 individuals per plant, or more than double that without alternative food! This indicates that this treatment promotes the installation of A. swirskii on this crop, and in fact improves the protection of this crop against the targets of this predator.

On a cucumber crop (FIG. 2), in the absence of alternative food, A. swirskii is not able to be maintained, the populations never exceed 5 individuals per plant, and these individuals come mainly from migration from adjacent plants. In the presence of A. ovatus eggs mixed with the acacia gum solution, rapid installation occurs, and in the month following the application up to 20 individuals per plant are observed (with an infestation of only 5 individuals per plant at DO). This figure thus indicates that in the presence of A. ovatus eggs in a sprayable but viscous composition a preventive treatment is finally possible with predatory mites on cucumber without having to deposit mini-colonies (sachets) regularly or to add predators by another means. The predator populations, with the treatment presented here, develop and install in a sustainable manner in the crop.

1. A sprayable aqueous composition comprising eggs of at least one arthropod species; and one or more thickeners and/or gelling agents of plant origin selected from the group consisting of plant gums, plant exudates, galactomannans derived from certain seeds, glucomannans, pectins located within cell walls, algae extracts, natural or modified starches, cellulose derivatives, gels derived from metabolic production of plants, and maltodextrins.

2. The composition according to claim 1, wherein said thickener(s) and/or gelling agent(s) are present in a quantity between 0.01 and 5% w/v.

3. The composition according to claim 1, wherein said thickener(s) and/or gelling agent(s) are present in a quantity that provides the composition with a dynamic viscosity at zero velocity gradient between 0.005 and 100 Pa·s.

4. The composition according to claim 1, wherein said arthropod eggs are members of one or more mite species, one or more insect species, or one or more crustacean species.

5. The composition according to claim 1, wherein said arthropod eggs are members of at least one mite species and at least one insect species, at least one mite species and at least one crustacean species, or at least one insect species and at least one crustacean species.

6. The composition according to claim 1, wherein said thickener(s) and/or gelling agent(s) are selected from the group consisting of acacia gum, carob gum, oat gum, guar gum, gum tragacanth, gum arabic, xanthan gum, karaya gum, tara gum, gellan gum, gum ghatti, konjac gum, and cassia gum.

7. The composition according to claim 1, wherein said thickener(s) and/or gelling agent(s) are selected from the group consisting of corn maltodextrins, tapioca maltodextrins, and potato maltodextrins.

8. A method for preparing a composition according to claim 1 comprising:
dissolving the thickener(s) and/or gelling agent(s) in an aqueous base, and
adding the arthropod eggs to this mixture.

9. The composition according to claim 1, wherein the composition is a nutrient source for biological control agents.

10. The composition according to claim 1, wherein the composition improves the effectiveness of biological control methods.

11. A biological control method comprising applying the composition according to claim 1 on a plant.

12. A biological control method comprising:
a) applying biological control predators or biological control compositions comprising the biological control predators, on plants;
b) applying the composition according to claim 1 on the plants.

13. The method according to claim 11 wherein the composition is sprayed on the plant.

14. The method according to claim 12 wherein the compositions are sprayed on the plants.

15. The composition according to claim 1 wherein the composition is a biological feed control agent.

16. The method for preparing a composition according to claim 8 wherein the aqueous base is water.
